(19) United States
(12) Patent Application Publication
(10) Pub. No.: US 2015/0079188 A1
(43) Pub. Date: Mar. 19, 2015

(54) Personal Care Formulation to Mitigate Vitamin D Deficiency

(71) Applicant: Nanoco Technologies Ltd., Manchester (GB)

(72) Inventors: Nathalie Gresty, Chester (GB); Nigel Pickett, Manchester (GB)

(21) Appl. No.: 14/484,854

(22) Filed: Sep. 12, 2014

Related U.S. Application Data
(60) Provisional application No. 61/877,803, filed on Sep. 13, 2013.

(51) Int. Cl. A61K 33/30; A61K 31/12; A61K 8/27; A61Q 17/04; A61K 8/35

(52) U.S. Cl. CPC A61K 33/30; A61Q 17/04; A61K 8/35; A61K 8/27; A61K 31/12
USPC 424/497; 424/59

(57) ABSTRACT

Herein, a personal care formulation is described. The formulation includes a personal care ingredient, in which quantum dots and a UVA-absorbing species are dissolved or dispersed. The formulation absorbs at least a portion of light at wavelengths below 280 nm and above 315 nm. In some embodiments, the formulation emits light with a peak maximum in the region of 290 nm to 300 nm. When applied to the skin, the formulation can be used to mitigate some of the harmful effects of sunlight and manmade lighting products, such as compact fluorescent lamps, while enabling the skin to absorb at least a portion of light required to synthesize vitamin D in vivo.

### BACKGROUND

1. Field of the Invention

The invention relates to a personal care formulation and more particularly to a personal care formulation that offers protection from ultraviolet (UV) rays but allows transmission of the wavelengths of radiation required for the synthesis of vitamin D in vivo. The personal care formulation comprises quantum dots.

2. Description of the Related Art

Increased awareness and understanding of the contribution of exposure to sunlight in causing skin cancer has led to the increased use of high sun protection factor (SPF) sunscreens. However, a concurrent increase in the number of instances of deficiency in vitamin D has been noted. Vitamin D is synthesised in the skin by the action of sunlight between the wavelengths of 280-315 nm, peaking between 295-297 nm, on the skin, [M. L. Stroud, Austr. Fam. Physician, 2008, 37, 1002; E. M. Hume, N. S. Lucas and H. H. Smith, Biochem. J., 1927, 21, 362; http://ods.od.nih. gov/factsheets/VitaminD-HealthProfessional/; http://www.nbcnews.com/id/4001172/]. Vitamin D promotes the absorption of calcium and phosphate from dietary sources, while also effecting the release of calcium from the bones. Deficiency can result in softening of the bones due to lack of calcium phosphate, leading to rickets in children and osteomalacia in adults. [L. Mervyn, Thorsons Complete Guide to Vitamins and Minerals; HarperCollins: London, 2000; pp. 93-94; http://www.nhs.uk/livewell/summerhealth/pages/vitamin-d-sunlight.aspx].

Of the UV rays emitted by sunlight, UVA and UVB are able to penetrate the ozone layer. With an emission range between 315-400 nm, UVA constitutes up to 95% of UV radiation reaching the surface of the Earth. UVA causes damage to cells in the basal layer of the skin's epidermis, where most skin cancers occur, but does not result in sunburn. Emission between 290-315 nm is defined as UVB. Exposure to UVB may cause in damage to the superficial layers of the epidermis, which can lead to sunburn and skin cancers. The overlap between the absorption spectrum of vitamin D and the UVB emission range is shown FIG. 1. The SPF of a sunscreen relates to the level of protection from sunburn-promoting UVB rays, but not UVA. Many high factor sunscreens do not provide adequate protection from UVA. Due to the high overlap between the absorption spectrum of vitamin D and the UVB emission spectrum, the higher the SPF, the greater the ability to reduce the level of photosynthesis of vitamin D in vivo as well as to prevent sun damage. While the majority of UVC rays (100-290 nm) emitted by sunlight are absorbed by the ozone layer, UVC, a mutagen and carcinogen that causes damage to collagen, is emitted by compact fluorescent lamps (CFLs). [T. Mironava, M. Hadjiargyrou, M. Simon and M. H. Rafailovich, Photochem. Photobio., 2012, 88, 1497].

In the case of vitamin D deficiency resulting from the use of sunscreens, one proposed solution is to increase dietary intake of vitamin D. However, in high quantities the vitamin can be toxic, [http://www.telegraph.co.uk/health/healthnews/9742176/Plethora-of-diseases-caused-by-low-vitamin-D.html] potentially leading to hypercalcemia and hypercalciuria. Further, dietary sources of vitamin D may not adequately correct a deficiency in patients with gastrointestinal malabsorption. In such cases, the benefits of dosed therapeutic exposure to UVB rays in promoting vitamin D synthesis by the skin may outweigh the health risks associated with UVB.

Due to the narrow absorption spectrum for the skin to produce vitamin D in vivo, a phototherapy approach, e.g. exposure to a lamp emitting between 280-315 nm and peaking around 295-297 nm, may be challenging without concurrent exposure to other wavelengths of undesired, harmful UV rays such as UVA and UVC.

Thus, there is a need for a therapeutic approach that allows the absorption of vitamin D-inducing radiation by the skin, but concurrently provides a protective barrier from other wavelengths of UV light such as UVA and UVC, to protect the skin from some of the damaging effects of sun and CFL exposure.

### SUMMARY

Herein, a topical personal care formulation is described. The formulation includes quantum dots (QDs) dispersed in a personal care ingredient such as an emollient, cream, or oil. The formulation may also include a UVA-absorbing species dissolved or dispersed in the personal care ingredient. Optionally, the QDs may be incorporated into a matrix material or encapsulated into beads prior to dissolution or dispersion in the personal care ingredient. The formulation absorbs at least a portion of light at wavelengths below 280 nm and above 315 nm. In some embodiments, the formulation emits light with a peak maximum in the region of 290 nm to 300 nm, i.e., within the “sweet spot” for vitamin D production.

### BRIEF DESCRIPTION OF THE DRAWINGS

FIG. 1 illustrates Absorption spectrum of vitamin D. The spectral range for UVB emission (290-320 nm) is also shown. [Adapted from FIG. 2 in D. Wolpowitz and B. A. Gilchrest, J. Am. Acad. Dermatol., 2006, 54, 301].

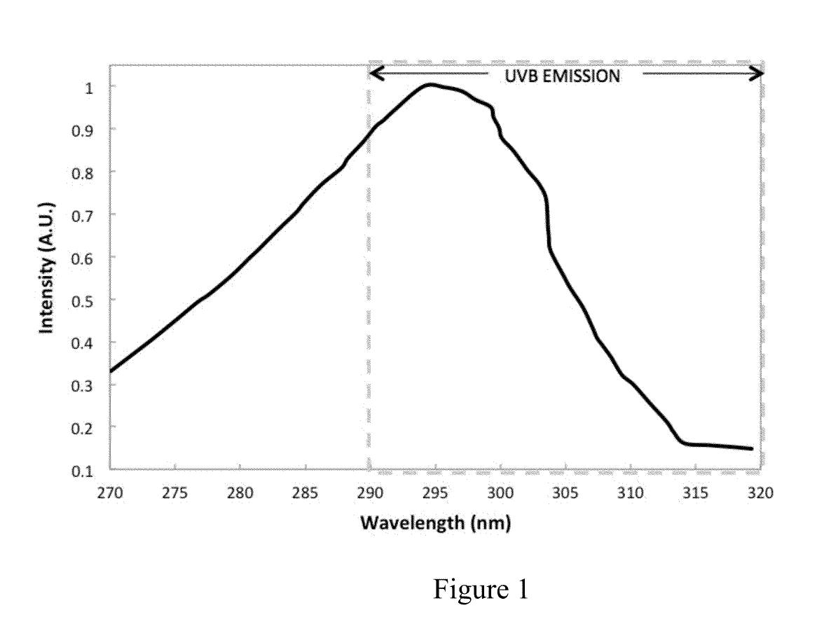

FIG. 2 shows a normalised simulated absorption spectrum of ZnO nanoparticles with an absorption maximum in the region of 263 nm, along with the normalised absorption spectra of vitamin D [adapted from FIG. 2 in D. Wolpowitz and B. A. Gilchrest, J. Am. Acad. Dermatol., 2006, 54, 301] and avobenzone. [Adapted from FIG. 5 in J. Boustie and P. Trouilles, J. Photochem. Photobio. B: Bio, 2012, 111, 17].

FIG. 3 shows a normalised simulated absorption and emission spectra of ZnO nanoparticles with an absorption maximum in the region of 278 nm and a photoluminescence maximum at 295 nm, along with the normalised absorption spectra of vitamin D [adapted from FIG. 2 in D. Wolpowitz and B. A. Gilchrest, J. Am. Acad. Dermatol., 2006, 54, 301] and avobenzone. [Adapted from FIG. 5 in J. Boustie and P. Trouilles, J. Photochem. Photobio. B: Bio, 2012, 111, 17].

### DESCRIPTION

Vitamin D is present in the body in two forms—Vitamin D₂ (D₂) and Vitamin D₃ (D₃)—both of which are not biologically active. The biologically active forms are the metabolites 25-hydroxyvitamin D, abbreviated 25(OH)D₂ and 25(OH)D₃, respectively. D₂ is obtained from dietary vegetables and supplements; D₃ is obtained from skin exposure to UVB radiation and from oily fish and vitamin D-fortified sources.

As mentioned above, vitamin D deficiency is associated with a number of maladies of the musculoskeletal system. Thus, vitamin D deficiency has been linked to symptoms, such as bone pain, myalgias, and generalized weakness. Vitamin D supplementation is generally indicated for patients with serum 25(OH)D levels of less than about 30 ng/mL. Most common supplementation is based on oral supplementation, either by changes in diet and/or by administration of oral vitamin D supplements. Oral supplementation can be hampered, however, by factors such as malabsorption, non-adherence, or inadequate dosing. Moreover, patients with maladies of the gastrointestinal tract, such as Crohn's disease, may have difficulty tolerating oral vitamin D supplementation.

For some patients, enhancement of cutaneous production of vitamin D is a more effective and reliable modality of treatment. However, cutaneous generation of vitamin D by exposure to UVB radiation may be accompanied by exposure to harmful UV rays such as UVA and UVC. As mentioned above, one root cause of vitamin D deficiency can be the use of high SPF sunscreen, which blocks the vitamin D-producing radiation in addition to harmful UV radiation.

Herein, a personal care formulation is described that, when applied to the human skin, mitigates the problems associated with vitamin D deficiency associated with high SPF sunscreens. By allowing a portion of UVB rays to reach the skin, while still absorbing UV rays at either side of the vitamin D absorption spectrum, the personal care formulation provides a relatively high degree of protection from the harmful effects on the skin of sunlight and manmade lighting products, such as CFLs.

The topical formulations described herein include quantum dots (QDs). QDs are luminescent nanoparticles of semiconductor material, with diameters typically in the range of 1 to 20 nm. Their photo-absorption and -luminescence can be tuned by manipulating the particle size. The unique optical and electronic properties of QDs originate from quantum confinement effects; as the QD diameter decreases the electron and hole wavefunctions become quantum confined, giving rise to discrete energy levels similar to those observed in atoms or molecules, resulting in an increase in the semiconductor band gap with decreasing QD diameter.

By decreasing the particle size, QDs can be tuned to absorb light at wavelengths shorter than the band gap (E_{g}) of their respective bulk semiconductor. Thus, QDs of wide, direct band gap semiconductors, such as ZnO (E_{g}=3.37 eV, 368 nm), hexagonal ZnS (E_{g}=3.91 eV, 317 nm) and Al₁₋ₓGaₓN (E_{g}=3.44-6.38 eV, 194-360 nm) can be manipulated to absorb in the middle ultraviolet (MUV, 200-300 nm) range. Further, as the particle size decreases, QD materials such as ZnO can become optically transparent, offering aesthetic advantages for personal care applications. QDs offer the same level of UV protection as larger particles commonly used in sunscreens, such as ZnO or TiO₂, but without leaving a white residue on the skin. The transparency of QD-containing formulations is further facilitated by the high absorption coefficient of QDs, enabling strong absorption (i.e., effectiveness) from a tiny amount of QD material.

Methods to synthesise QDs are well known in the prior art. Of the methods previously described, colloidal syntheses, such as hot-injection and molecular seeding methods, can be used to produce QDs with a homogeneous morphology and size distribution, leading to a well-defined absorption profile. Further, colloidal QDs are capped with organic ligands that impart solubility in a range of media, facilitating processability. Examples of QD synthesis are described in U.S. Pat. Nos. 6,322,901; 7,803,423; 7,985,446; and 8,062,703. The most widely studied QDs are based on II-VI semiconductor materials. Examples include CdS, CdSe, and the like. The '901 Patent referenced here is directed to such QDs. Due to negative health and environmental issues associated with heavy metals, such as Cd, it is preferred that the QDs for topical application do not include heavy metals. The '423, '446, and '703 Patents discuss the synthesis of such Cd-free QDs on a commercial scale. Examples of suitable metal-oxide QDs for use as described herein are described in U.S. patent application Ser. No. 14/483,870 filed Sep. 11, 2014 under attorney docket number 038-0088US, the entire contents of which are incorporated herein by reference.

The formulations described herein incorporate QDs with an absorption wavelength that can be precisely tuned by manipulating the particle size. For example, the QDs can preferentially absorb light below 280 nm and transmit light above 280 nm. By selecting QDs of an appropriate particle size and in some embodiments combining the QDs with one or more other UV-absorbing species, such as organic molecules, inorganic particulates or organic particulates, the personal care formulation can be tailored to absorb across the UV range, while leaving a window between ˜280-315 nm where the absorption is relatively reduced to thus facilitate vitamin D production during exposure of the skin to sunlight and/or manmade emitters of UV light. The formulation effectively acts as a “band pass filter,” selectively passing radiation that stimulates the cutaneous production of vitamin D.

In some embodiments, within window for vitamin D absorption the personal care formulation provides a degree of protection from UVB rays, but the formulation's absorption of UVB rays is relatively lower than for UVA and UVC rays. Thus, the formulation allows a portion of UVB light to penetrate the skin to facilitate the photosynthesis of vitamin D in vivo, while offering stronger protection from UVA and UVC rays. Preferably, the formulation absorbs relatively strongly at wavelengths less than 280 nm and greater than 315 nm, and relatively weakly between 280-315 nm. For example, the formulation's absorbance at 300 nm may be 50%, 25%, 10%, 5%, or 1% or less of the absorbance at 280 nm.

In alternative embodiments, the QDs absorb light at wavelengths below the vitamin D absorption window and re-emit within the window, to act as a topical phototherapy formulation to promote vitamin D synthesis, while offering protection from UVA and UVC rays. Preferably, the formulation absorbs strongly at wavelengths less than 280 nm and greater than 315 nm, relatively weakly between 280-315 nm, and emits light with a peak maximum in the region of 290-300 nm, most preferably around 295 nm.

The preparation of a personal care formulation comprising QDs has previously been described in the applicant's granted U.S. Pat. No. 7,341,734, the entire contents of which are herein incorporated by reference. QDs are dispersed into a personal care ingredient, either directly, or by first incorporating the nanoparticles into a matrix material that is subsequently micronized prior to dissolution or dispersion in the personal care ingredient. As used herein, the term “dispersed” refers to either dissolved or dispersed.

Suitable personal care ingredients include, but are not restricted to emollients, creams, or oils. Examples include fatty acids, fatty esters, waxes, oils, triglycerides, long chain alcohols, silicones, emulsions (e.g. water and oil, oil and wax, wax and water), antiseptics, astringents, and combinations thereof. Suitable matrix materials may include, but are not restricted to, silica sol-gels, polystyrenes, silicon-based polymers, polyacrylates, polyurethanes, polycarbonates, and combinations thereof. Preferably, the absorption spectrum of any personal care ingredient and/or matrix material used in the formulation should fall substantially outside of the skin's vitamin D absorption window (280-315 nm).

As an alternative to encapsulating the QDs in a bulk matrix material that is subsequently micronized, in further embodiments the QDs may be encapsulated into a plurality of discrete beads prior to dissolution or dispersion into the personal care ingredient. The encapsulation method serves to protect the nanoparticles from the surrounding physical environment and/or processing conditions, thus improving the QD stability. Encapsulation of the QDs, for example within a polymer to form QD beads, may also serve to prevent any chemical reaction of the nanoparticles in vivo. Examples of the encapsulation of QDs are described in the applicant's co-pending U.S. Patent Application Publication Nos. 2010/0113813, and 2011/0068321, the contents of which are incorporated herein by reference. The encapsulation medium should be optically clear. The absorption spectrum of any encapsulation medium should fall substantially outside of the skin's vitamin D absorption window (280-315 nm). In some embodiments, the encapsulation medium for the QD beads includes, but is not restricted to, a resin, polymer, monolith, sol-gel, epoxy, silicone, (meth)acrylate. According to some embodiments, the QDs may be encapsulated within primary beads which are then encapsulated within secondary beads, providing a bead-in-bead structure, as described in U.S. Patent Application Publication No. 2011/0068321. According to some embodiments, the QDs can be encapsulated within beads that include a surface coating applied to the beads. Examples of surface coatings include polymers or inorganic materials such as Al₂O₃ or other metal oxides.

In some embodiments, the QD material absorbs strongly in the UV region below 280 nm, but is weakly or non-emissive. Any suitable semiconductor material may be used to fabricate QDs with a band gap of 4.43 eV or greater. One skilled in the art will recognise that the required band gap (i.e. particle size) to produce QDs absorbing below 280 nm will depend on the absorption profile of the nanoparticles. In preferred embodiments, the QDs display a sharp absorption edge with an absorption maximum in the range 260±10 nm. Suitable QD material includes, but is not restricted to, BN, AlN, GaN, ZnO, ZnS, and SnO, including doped species and alloys thereof.

In other embodiments, the QD material absorbs strongly in the UV region below 280 nm and re-emits in the region of 295 nm. Any suitable semiconductor material may be used to fabricate QDs with a band gap of 4.43 eV or greater and a Stokes shift of 15 nm or more. One skilled in the art will recognise that the required band gap (i.e. particle size) to produce QDs absorbing below 280 nm and re-emitting at ˜295 nm will depend on the absorption profile and the Stokes shift of the nanoparticles. In preferred embodiments, the QDs display a sharp absorption edge with an absorption maximum in the range 260±10 nm and a photoluminescence maximum (PLₘₐₓ) in the region of 295±5 nm. Organically-capped colloidal QD cores generally display a low photoluminescence quantum yield (QY), due to exciton recombination via surface defects and dangling bonds. Modification of the structural and electronic architecture of the QDs, while maintaining control of the size-tuneable band gap, can be achieved via the epitaxial growth of one or more “shell” layers of different band gap semiconductor material(s) on the nanoparticle surface. A core/shell architecture is achieved by the growth of a wider band gap material on the core surface, e.g. ZnO/ZnS. Shelling serves to eliminate surface defects and dangling bonds, to significantly improve the QY and enhance stability by suppressing interactions between charge carriers and the surrounding environment. Further improvements in stability can be achieved with additional shelling layers, as in the core/multishell structure, e.g. GaN/BN/AlN, a quantum dot-quantum well architecture, e.g. ZnS/ZnO/ZnS, or a core/compositionally graded shell structure, e.g. ZnO/Zn₁₋ₓAlₓN_{1-y}O_{y}. As used herein, the term core/shell QDs refers to QDs having a core and one or more “shells,” as well as to compositionally graded architectures.

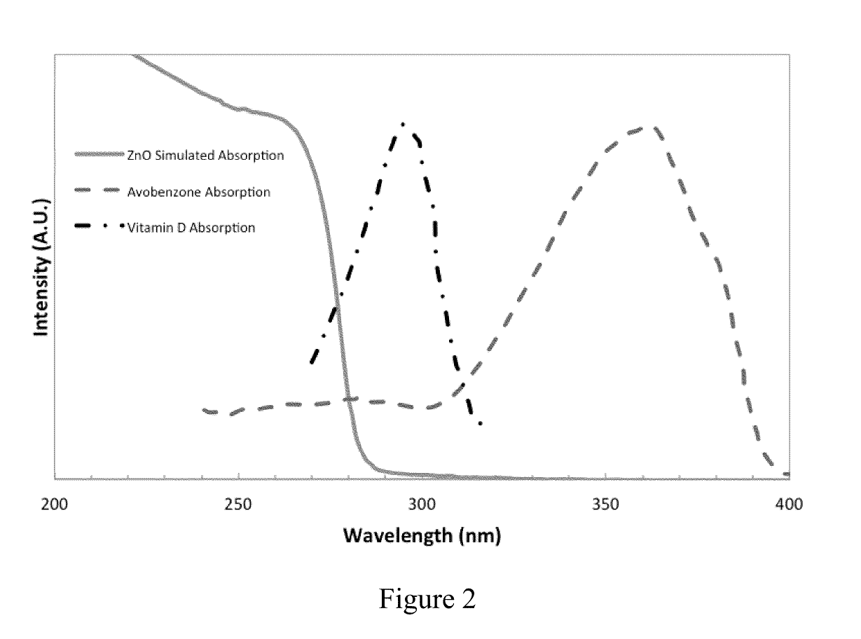

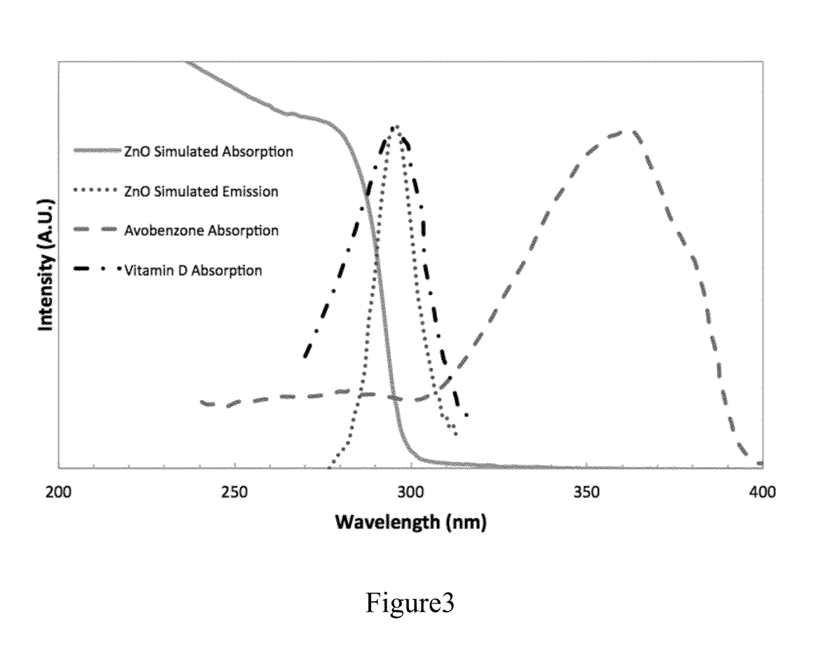

In some embodiments, the UVC-absorbing QDs are combined with one or more non-QD, UVA-absorbing species. Any suitable UVA-absorbing species that can be dissolved or dispersed in the personal care ingredient may be used, providing its absorption spectrum lies substantially outside of the skin's vitamin D absorption window (280-315 nm), and may include an organic chemical, inorganic particulates, organic particulates, and combinations thereof. In particular embodiments, the UVA-absorbing species is avobenzone, the absorption spectrum of which is shown in FIG. 2 and FIG. 3. Alternatively, or in addition, the UVA-absorbing species can be ecamsule, bisdisulizole disodium, diethylamino hydroxybenzoyl hexyl benzoate, or menthyl anthranilate.

### EXAMPLES

The QD absorption and emission spectra provided in the following examples have been simulated for the purpose of illustrating the methods described herein.

### Example 1

### Personal Care Formulation Comprising Non-Emissive UVC-Absorbing ZnO Quantum Dots and Avobenzone

The personal care formulation comprises ZnO QDs with an absorption peak in the region of 263 nm, but where the nanoparticles are relatively non-emissive. The QDs are combined with avobenzone, a UVA-absorbing species, in a desired ratio, and appropriate personal care ingredients to formulate a personal care product that absorbs substantially in the UVA and UVC regions of the electromagnetic spectrum, but to a lesser extent in the region for vitamin D absorption. The normalised absorption spectra of avobenzone and vitamin D, along with the normalised simulated absorption spectrum of ZnO QDs (UV_{abs}˜263 nm), are shown in FIG. 2. The UV absorption spectrum of the personal care formulation can be tailored by manipulating the relative concentrations of the ZnO QDs and avobenzone within the personal care ingredient.

### Example 2

### Personal Care Formulation Comprising UVC-Absorbing, UVB-Emitting ZnO Quantum Dots and Avobenzone

The personal care formulation comprises ZnO QDs with an absorption peak in the region of 278 nm and a photoluminescence maximum at 295 nm. The QDs are combined with avobenzone, a UVA absorber, in a desired ratio, and appropriate personal care ingredients to formulate a personal care product that absorbs substantially in the UVA and UVC regions of the electromagnetic spectrum, but to a lesser extent in the region for vitamin D absorption. Further, the ZnO nanoparticles re-emit within the vitamin D absorption window, to promote the photosynthesis of vitamin D. The normalised absorption spectra of avobenzone and vitamin D, along with the normalised simulated absorption and emission spectra of ZnO QDs (UV_{abs}˜278 nm; PLₘₐₓ=295 nm), are shown in FIG. 3. The UV absorption spectrum of the personal care formulation can be tailored by manipulating the relative concentrations of the ZnO QDs and avobenzone within the personal care ingredient.

The foregoing description of preferred and other embodiments is not intended to limit or restrict the scope or applicability of the inventive concepts conceived of by the Applicants. It will be appreciated with the benefit of the present disclosure that features described above in accordance with any embodiment or aspect of the disclosed subject matter can be utilized, either alone or in combination, with any other described feature, in any other embodiment or aspect of the disclosed subject matter.

1. A formulation comprising:
a personal care ingredient;
quantum dots (QDs) that preferentially absorb light at wavelengths below 280 nm and transmit light above 280 nm; and
a UVA-absorbing species; wherein
the QDs and the UVA-absorbing species are dispersed in the personal care ingredient.

2. The formulation of claim 1, having an absorbance at 300 nm that is 10% or less than the absorbance at 280 nm.

3. The formulation of claim 1, wherein the QDs emit light with a maximum emission within the region from about 290 nm to about 300 nm.

4. The formulation of claim 1, wherein the personal care ingredient comprises a fatty acid, fatty ester, wax, oil, triglyceride, long chain alcohol, silicone, emulsion, antiseptic, astringent, or combination thereof

5. The formulation of claim 1, wherein the quantum dots have an absorption maximum in the region of 250 nm to 270 nm.

6. The formulation of claim 1, wherein the QDs comprise BN, AlN, GaN, ZnO, ZnS, SnO, or doped species or alloys thereof.

7. The formulation of claim 1, wherein the quantum dots display a Stokes shift of about 15 nm or more.

8. The formulation of claim 1, wherein the QDs are core/shell QDs.

9. The formulation of claim 1, wherein the UVA-absorbing species is an organic compound, an inorganic particulate material or an organic particulate material.

10. The formulation of claim 1, wherein the UVA-absorbing species is avobenzone, ecamsule, bisdisulizole disodium, diethylamino hydroxybenzoyl hexyl benzoate, or menthyl anthranilate.

11. The formulation of claim 1, wherein the QDs are encapsulated within a matrix material that is dispersed in the personal care ingredient.

12. The formulation of claim 11, wherein the matrix material comprises beads.

13. The formulation of claim 12, wherein the beads comprise an inorganic surface coating.

14. A method of treating vitamin D deficiency, comprising administering to a patient a topical formulation comprising:
a personal care ingredient;
quantum dots (QDs) that preferentially absorb light at wavelengths below 280 nm and transmit light above 280 nm; and
a UVA-absorbing species; wherein
the QDs and the UVA-absorbing species are dispersed in the personal care ingredient.

15. The method of claim 14, wherein the formulation has an absorbance at 300 nm that is 10% or less than the absorbance at 280 nm.

16. The method of claim 14, wherein the QDs emit light with a maximum emission within the region from about 290 nm to about 300 nm.

17. The method of claim 14, wherein the QDs comprise BN, AlN, GaN, ZnO, ZnS, SnO, or doped species or alloys thereof

18. The method of claim 14, further comprising monitoring the serum 25(OH)D level of the patient and continuing the administering until the serum 25(OH)D level is greater than about 30 ng/mL.

19. A formulation comprising:
a personal care ingredient;
a substantially transparent matrix material comprising quantum dots (QDs) encapsulated within the matrix material, the QDs capable of preferentially absorbing light at wavelengths below 280 nm and transmitting light above 280 nm; and
a UVA-absorbing species; wherein
the matrix material and the UVA-absorbing species are dispersed in the personal care ingredient.

20. The formulation of claim 19, wherein the QDs comprise BN, AlN, GaN, ZnO, ZnS, SnO, or doped species or alloys thereof.

21. The formulation of claim 19, wherein the matrix material comprises silica, a sol-gel, polystyrene, a silicon-based polymer, polyacrylate, polyurethane, polycarbonate, or combinations thereof.

22. The formulation of claim 19, wherein the UVA-absorbing species is avobenzone, ecamsule, bisdisulizole disodium, diethylamino hydroxybenzoyl hexyl benzoate, or menthyl anthranilate.

23. The formulation of claim 19, wherein the personal care ingredient comprises a fatty acid, fatty ester, wax, oil, triglyceride, long chain alcohol, silicone, emulsion, antiseptic, astringent, or combination thereof.
